(19) United States
(12) Patent Application Publication
(10) Pub. No.: US 2025/0186206 A1
(43) Pub. Date: Jun. 12, 2025

(54) DELIVERY APPARATUS FOR A MECHANICALLY EXPANDABLE PROSTHETIC HEART VALVE

(71) Applicant: EDWARDS LIFESCIENCES CORPORATION, Irvine, CA (US)

(72) Inventors: Anatoly Dvorsky, Haifa (IL); Ilan Leshecz, Hadera (IL); Ofir Witzman, Harish (IL)

(73) Assignee: EDWARDS LIFESCIENCES CORPORATION, Irvine, CA (US)

(21) Appl. No.: 19/062,393

(22) Filed: Feb. 25, 2025

Related U.S. Application Data
(63) Continuation of application No. PCT/US2023/030915, filed on Aug. 23, 2023.
(60) Provisional application No. 63/402,428, filed on Aug. 30, 2022.

(51) Int. Cl. A61F 2/24

(52) U.S. Cl. CPC A61F 2/243; A61F 2220/0025

(57) ABSTRACT

A delivery apparatus for implanting a prosthetic valve is disclosed. The prosthetic valve can comprise a mechanically actuatable frame comprising at least one actuator member, the actuator member configured to be rotated to radially expand or compress the frame. The delivery apparatus can comprise at least one actuator assembly configured to actuate the actuator member of the prosthetic valve, the actuator assembly comprising an outer sleeve member having a lumen and an inner driver member coaxially disposed within the lumen of the sleeve member, the driver member being rotatable relative to the sleeve member, wherein the actuator assembly is configured to be transitioned between an engaged state and a disengaged state with the prosthetic valve, and wherein the driver member is configured to be selectively magnetically coupled to the actuator member in the engaged state.

### CROSS-REFERENCE TO RELATED APPLICATIONS

This application is a continuation of PCT Application No. PCT/US2023/030915, filed on Aug. 23, 2023, which claims the benefit of U.S. Provisional Application No. 63/402,428, filed Aug. 30, 2022, both of which are incorporated by reference herein.

### FIELD

The present disclosure relates to apparatus and methods for delivering, expanding, implanting, and deploying implantable, radially expandable prosthetic devices, such as mechanically expandable prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Mechanically expandable prosthetic heart valves can provide one or more advantages over self-expandable and balloon-expandable prosthetic heart valves. For example, mechanically expandable prosthetic heart valves can be expanded to various working diameters. Mechanically expandable prosthetic heart valves can also be compressed after an initial expansion (for example, for repositioning and/or retrieval). Mechanically expandable prosthetic valves typically have one or more delivery device actuators that are coupled to respective actuators of the prosthetic valve. During an implantation procedure, the delivery device actuators apply forces to the actuators of the prosthetic valve to produce radial expansion, and optionally radial compression of the prosthetic valve. After the prosthetic valve is expanded at the desired implantation site within a patient, the delivery device actuators are de-coupled from the prosthetic valve and the delivery device is removed from the patient's body.

Despite the recent advancements in percutaneous valve technology, there remains a need for improved transcatheter heart valves and delivery devices for such valves.

### SUMMARY

Described herein are prosthetic heart valves, delivery apparatuses, and methods for implanting prosthetic heart valves. The disclosed delivery apparatus and methods can, for example, improve the positioning and/or alignment between an actuator assembly of the delivery apparatus and a mechanical actuator of a prosthetic heart valve. The disclosed delivery apparatuses can selectively induce a magnetic engagement between the actuator assembly and the actuator during an implantation procedure to simplify engagement therebetween. As such, the devices and methods disclosed herein can, among other things, overcome one or more of the deficiencies of typical delivery apparatuses for mechanically expandable prosthetic valves.

In some examples, a delivery apparatus for implanting a prosthetic valve comprising a mechanically actuatable frame comprising at least one actuator member, the actuator member configured to be rotated to radially expand or compress the frame, the delivery apparatus comprises at least one actuator assembly.

In some aspects, the at least one actuator assembly is configured to actuate the actuator member of the prosthetic valve.

In some aspects, the actuator assembly comprises an outer sleeve member having a lumen and an inner drive member coaxially disposed within the lumen of the sleeve member.

In some aspects, the driver member is rotatable relative to the sleeve member.

In some aspects, the actuator assembly is configured to be transitioned between an engaged state and a disengaged state with the prosthetic valve.

In some aspects, the driver member is configured to be selectively magnetically coupled to the actuator member in the engaged state.

In some aspects, the driver member includes an electromagnet configured to induce an electromagnetic field.

In some aspects, the delivery apparatus comprises a handle coupled to the at least one actuator assembly, wherein the handle comprises an input device operatively coupled to the driver member, wherein the input device is configured to magnetize and demagnetize the electromagnet.

In some aspects, the delivery apparatus comprises one or more wires electrically operatively coupled to the electromagnet and the input device, wherein the one or more wires extend through a lumen of the driver member.

In some aspects, the handle comprises one or more visual indicators to indicate whether the electromagnet is magnetized or demagnetized and/or whether the at least one actuator assembly is in the engaged state or the disengaged state.

In some examples, a delivery apparatus for implanting a prosthetic valve comprising a mechanically actuatable frame comprising at least one actuator member, the actuator member configured to be rotated to radially expand or compress the frame, the delivery apparatus comprises at least one actuator assembly configured to actuate the actuator member of the prosthetic valve, the actuator assembly comprising an outer sleeve member having a lumen and an inner driver member coaxially disposed within the lumen of the sleeve member, the driver member being rotatable relative to the sleeve member, wherein the actuator assembly is configured to be transitioned between an engaged state and a disengaged state with the prosthetic valve, and wherein the driver member is configured to be selectively magnetically coupled to the actuator member in the engaged state.

In some examples, a delivery apparatus for a prosthetic valve comprises a handle; and a driver having a proximal end portion coupled to the handle and a distal end portion comprising an electromagnet configured to releasably and magnetically couple the driver to an actuator of the prosthetic valve.

In some examples, an assembly comprises a prosthetic valve comprising a frame and at least one actuator coupled to the frame, the actuator operable to move the frame between a radially expanded configuration and a radially compressed configuration; and at least one actuator assembly configured to induce an electromagnetic field, wherein the electromagnetic field releasably couples the at least one actuator assembly to the at least one actuator.

In some examples, an assembly comprises a prosthetic valve having a radially expandable and compressible frame, a valvular structure disposed within the frame and configured to regulate a flow of blood through the frame in one direction, and at least one actuator operatively coupled to the frame, wherein the actuator is configured to be rotated, via rotation of a head thereof, to radially expand or compress the frame; and a delivery device comprising a driver member configured to form a releasable electromagnetic connection with the actuator and rotate the actuator upon rotation of the driver member.

In some examples, a delivery apparatus for a prosthetic valve comprising a mechanically actuatable frame comprising a plurality of actuator members, the actuator members configured to be rotated to radially expand or compress the frame, the delivery apparatus comprises a plurality of actuator assemblies configured to rotate the actuator members of the prosthetic valve, each actuator assembly comprising a rotatable driver member and a sensor electrically coupled to the driver member, wherein the sensor is configured to detect a resistive force exerted by the actuator member on the driver member during rotation.

In some examples, an assembly comprises a prosthetic valve comprising a frame and at least one actuator coupled to the frame, the actuator operable to move the frame between a radially expanded configuration and a radially compressed configuration; and a plurality of actuator assemblies, wherein each actuator assembly comprises a driver member configured to rotate the actuator upon rotation of the driver member and a sensor electrically coupled to the driver member, the sensor configured to detect a resistive force exerted by the actuator on the driver member to resist rotation.

In some examples, a method of implanting a prosthetic valve comprises magnetically coupling a driver of an actuator assembly of a delivery device to an actuator of the prosthetic valve; delivering the prosthetic valve to an implantation location within a patient's body; radially expanding the prosthetic valve to a functional size; and releasing the driver from the actuator.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of one example of a prosthetic valve including a frame and a plurality of leaflets attached to the frame.

FIG. 1B is a perspective view of the prosthetic valve of FIG. 1A with an outer skirt disposed around the frame.

FIG. 2A is a perspective view of a frame for the prosthetic valve of FIG. 1A.

FIG. 2B is a front portion of the frame shown in FIG. 2A.

FIG. 3 is a side elevation view of a delivery apparatus for a prosthetic device, such as a prosthetic valve, according to one example.

FIG. 4 is a detailed perspective view of an apex of the frame of FIG. 2A.

FIG. 5 is a detailed perspective view of the apex of the frame coupled to an actuator assembly of a delivery device, according to one example.

FIG. 6 is a perspective view of an outer layer of the actuator assembly of FIG. 5.

FIG. 7 is a perspective view of an inner layer of the actuator assembly of FIG. 5.

FIG. 8 is a perspective view of an actuator of the frame of FIG. 2A.

FIG. 9 is a cross-sectional view of the actuator of FIG. 8 coupled to the actuator assembly of FIG. 5.

FIG. 10 is a perspective view of an outer layer of an actuator assembly, according to one example.

FIG. 11 is a perspective view of an inner layer of an actuator assembly, according to one example.

FIG. 12 is a cross-sectional view of the actuator of FIG. 8 coupled to the outer and inner layers of FIGS. 10 and 11.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can include one or more expansion mechanisms that can be actuated using the delivery apparatus to expand the prosthetic valve to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

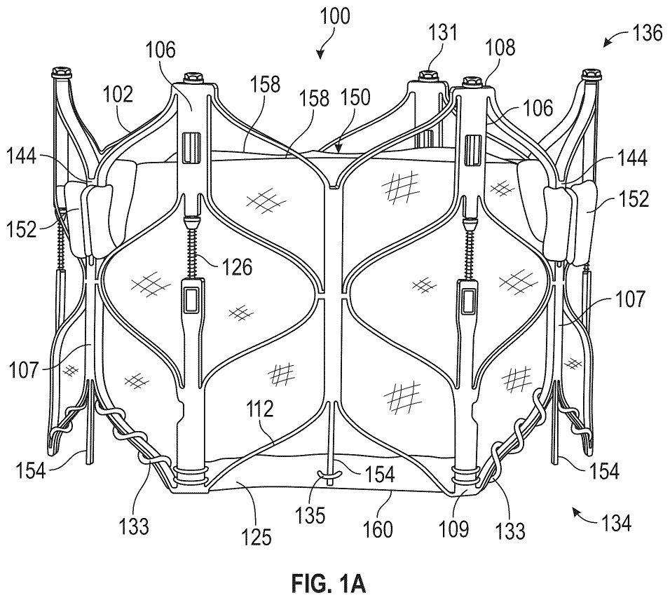

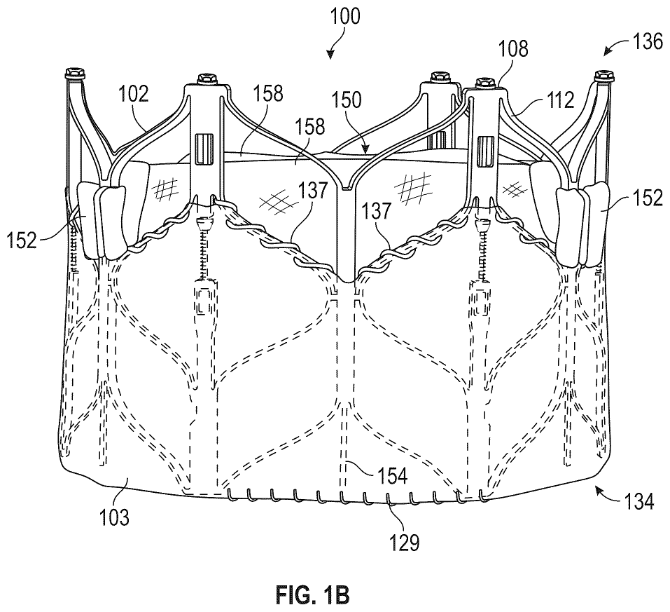

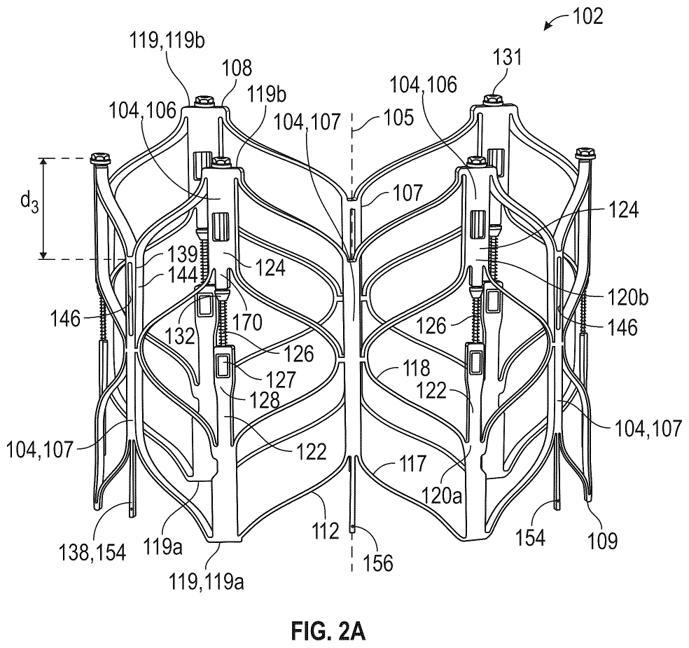

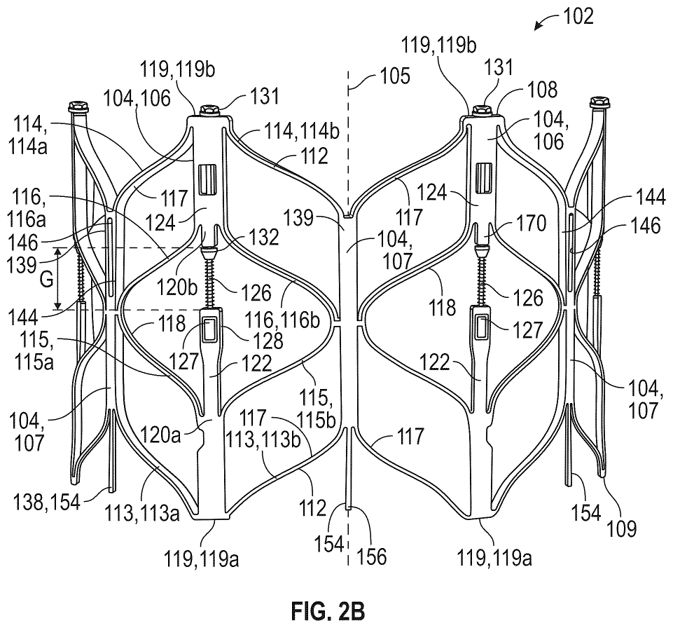

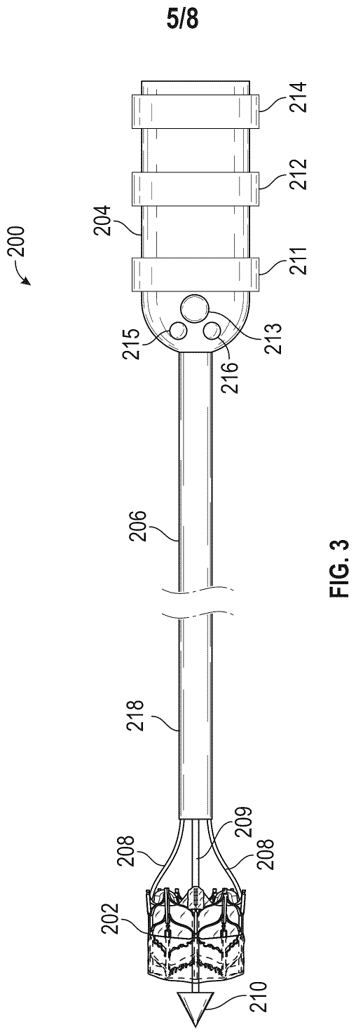

FIGS. 1A-2B illustrate an example of a prosthetic device (for example, prosthetic heart valve) that can be advanced through a patient's vasculature, such as to a native heart valve, by a delivery apparatus, such as the delivery apparatus shown in FIG. 3. The frame of the prosthetic heart valve can include one or more mechanical expansion and locking mechanisms that can be integrated into the frame-specifically, into axially extending posts of the frame. The mechanical expansion and/or locking mechanisms can be removably coupled to, and/or actuated by, the delivery apparatus to radially expand the prosthetic heart valve and lock the prosthetic heart valve in one or more radially expanded states. For example, the delivery apparatus and the mechanical expansion and/or locking mechanisms can be magnetically coupled together during radial expansion of the prosthetic heart valve. The magnetic engagement between the delivery apparatus and mechanical expansion and/or locking mechanisms can decrease the likelihood of misalignment of these components during an implantation procedure. Further, the mating components of the delivery device and the prosthetic valve are easier to manufacture than known mechanisms.

### Examples of the Disclosed Technology

FIGS. 1A-2B show a prosthetic valve 100, according to one example. Any of the prosthetic valves disclosed herein are adapted to be implanted in the native aortic annulus, although in some examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In some example, the disclosed prosthetic valves can be implanted within a docking device implanted within or at the native mitral valve, such as disclosed in PCT Publication No. WO2020/247907, which is incorporated herein by reference. In some example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

FIGS. 1A-2B illustrate an example of a prosthetic valve 100 (which also may be referred to herein as “prosthetic heart valve 100”) having a frame 102. FIGS. 2A-2B show the frame 102 by itself, while FIGS. 1A-1B show the frame 102 with a valvular structure 150 (which can comprise leaflets 158, as described further below) mounted within and to the annular frame 102. FIG. 1B additionally shows an optional skirt assembly comprising an outer skirt 103. While only one side of the frame 102 is depicted in FIG. 2B, it should be appreciated that the frame 102 forms an annular structure having an opposite side that is substantially identical to the portion shown in FIG. 1B, as shown in FIGS. 1A-2A.

As shown in FIGS. 1A and 1B, the valvular structure 150 is coupled to and supported inside the frame 102. The valvular structure 150 is configured to regulate the flow of blood through the prosthetic valve 100, from an inflow end portion 134 to an outflow end portion 136. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form commissures 152, each of which can be secured to a respective commissure support structure 144 (also referred to herein as “commissure supports”) and/or to other portions of the frame 102, as described in greater detail below.

In the example depicted in FIGS. 1A and 1B, the valvular structure 150 includes three leaflets 158, which can be arranged to collapse in a tricuspid arrangement. Each leaflet 158 can have an inflow edge portion 160 (which can also be referred to as a cusp edge portion) (FIG. 1A). The inflow edge portions 160 of the leaflets 158 can define an undulating, curved scallop edge that generally follows or tracks portions of struts 112 of frame 102 in a circumferential direction when the frame 102 is in the radially expanded configuration. The inflow edge portions 160 of the leaflets 158 can be referred to as a “scallop line.”

The prosthetic valve 100 may include one or more skirts mounted around the frame 102. For example, as shown in FIG. 1B, the prosthetic valve 100 may include an outer skirt 103 mounted around an outer surface of the frame 102. The outer skirt 103 can function as a sealing member for the prosthetic valve 100 by sealing against the tissue of the native valve annulus and helping to reduce paravalvular leakage past the prosthetic valve 100. In some cases, an inner skirt (not shown) may be mounted around an inner surface of the frame 102. The inner skirt can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic valve 100. In some examples, the inflow edge portions 160 of the leaflets 158 can be sutured to the inner skirt generally along the scallop line. The inner skirt can in turn be sutured to adjacent struts 112 of the frame 102. In some examples, as shown in FIG. 1A, the leaflets 158 can be sutured directly to the frame 102 or to a reinforcing member 125 (also referred to as a reinforcing skirt or connecting skirt) in the form of a strip of material (for example, a fabric strip) which is then sutured to the frame 102, along the scallop line via stitches (for example, whip stitches) 133.

The inner and outer skirts and the connecting skirt 125 can include any of various suitable biocompatible materials, including any of various synthetic materials, including fabrics (for example, polyethylene terephthalate fabric) or natural tissue (for example, pericardial tissue). Further details regarding the use of skirts or sealing members in prosthetic valve can be found, for example, in U.S. Patent Publication No. 2020/0352711, which is incorporated herein by reference.

Further details regarding the assembly of the leaflet assembly and the assembly of the leaflets and the skirts to the frame can be found, for example, in U.S. Provisional Application Nos. 63/209,904, filed Jun. 11, 2021, and 63/224,534, filed Jul. 22, 2021, which are incorporated herein by reference. Further details of the construction and function of the frame 102 can be found in International Patent Application No. PCT/US2021/052745, filed Sep. 30, 2021, which is incorporated herein by reference.

The frame 102, which is shown alone and in greater detail in FIGS. 2A and 2B, comprises and inflow end 109, an outflow end 108, and a plurality of axially extending posts 104. The axial direction of the frame 102 is indicated by a longitudinal axis 105, which extends from the inflow end 109 to the outflow end 108 (FIGS. 2A and 2B). Some of the posts 104 can be arranged in pairs of axially aligned first and second struts or posts 122, 124. An actuator 126 (such as the illustrated threaded rod or bolt) can extend through one or more pairs of posts 122, 124 to form an integral expansion and locking mechanism or actuator mechanism 106 configured to radially expand and compress the frame 102, as further described below. One or more of posts 104 can be configured as support posts 107.

The actuator mechanisms 106 (which can be used to radially expand and/or radially compress the prosthetic valve 100) can be integrated into the frame 102 of the prosthetic valve 100, thereby reducing the crimp profile and/or bulk of the prosthetic valve 100. Integrating the actuator mechanisms 106 (which can also be referred to herein as “expansion and locking mechanisms”) into the frame 102 can also simplify the design of the prosthetic valve 100, making the prosthetic valve 100 less costly and/or easier to manufacture. In the illustrated example, an actuator 126 extends through each pair of axially aligned posts 122, 124. In some examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As one example, the prosthetic valve 100 can include equal numbers of support posts 107 and pairs of actuator posts 122, 124 and the pairs of posts 122, 124 and the support posts 107 can be arranged in an alternating order such that each strut 112 is positioned between one of the pairs of posts 122, 124 and one of the support posts 107 (that is, each strut 112 can be coupled on one end to one of the posts 122, 124 and can be coupled on the other end to one of the support posts 107). However, the prosthetic valve 100 can include different numbers of support posts 107 and pairs of posts 122, 124 and/or the pairs of posts 122, 124 and the support posts 107 can be arranged in a non-alternating order, in some examples.

As illustrated in FIG. 2B, the struts 112 can include a first row of struts 113 at or near the inflow end 109 of the prosthetic valve 100, a second row of struts 114 at or near the outflow end 108 of the prosthetic valve 100, and third and fourth rows of struts 115, 116, respectively, positioned axially between the first and second rows of struts 113, 114. The struts 112 can form and/or define a plurality of cells (that is, openings) in the frame 102. For example, the struts 113, 114, 115, and 116 can at least partially form and/or define a plurality of first cells 117 and a plurality of second cells 118 that extend circumferentially around the frame 102. Specifically, each first cell 117 can be formed by two struts 113a, 113b of the first row of struts 113, two struts 114a, 114b of the second row of struts 114, and two of the support posts 107. Each second cell 118 can be formed by two struts 115a, 115b of the third row of struts 115 and two struts 116a, 116b of the fourth row of struts 116. As illustrated in FIGS. 2A and 2B, each second cell 118 can be disposed within one of the first cells 117 (that is, the struts 115a-116b forming the second cells 118 are disposed between the struts forming the first cells 117 (that is, the struts 113a, 113b and the struts 114a, 114b), closer to an axial midline of the frame 102 than the struts 113a-114b).

As illustrated in FIGS. 2A and 2B, the struts 112 of frame 102 can comprise a curved shape. Each first cell 117 can have an axially-extending hexagonal shape including first and second apices 119 (for example, an inflow apex 119a and an outflow apex 119b). In examples where the delivery apparatus is releasably connected to the outflow apices 119b (as described below), each inflow apex 119a can be referred to as a “distal apex” and each outflow apex 119b can be referred to as a “proximal apex”. Each second cell 118 can have a diamond shape including first and second apices 120 (for example, distal apex 120a and proximal apex 120b). In some examples, the frame 102 comprises six first cells 117 extending circumferentially in a row, six second cells 118 extending circumferentially in a row within the six first cells 117, and twelve posts 104. However, in some examples, the frame 102 can comprise a greater or fewer number of first cells 117 and a correspondingly greater or fewer number of second cells 118 and posts 104.

As noted above, some of the posts 104 can be arranged in pairs of first and second posts 122, 124. The posts 122, 124 are aligned with each other along the length of the frame 102 and are axially separated from one another by a gap G (FIG. 2B) (those with actuators 126 can be referred to as actuator posts or actuator struts). Each first post 122 (that is, the lower post shown in FIGS. 2A and 2B) can extend axially from the inflow end 109 of the prosthetic valve 100 toward the second post 124, and the second post 124 (that is, the upper post shown in FIGS. 2A and 2B) can extend axially from the outflow end 108 of the prosthetic valve 100 toward the first post 122. For example, each first post 122 can be connected to and extend from an inflow apex 119a and each second post 124 can be connected to and extend from an outflow apex 119b. Each first post 122 and the second post 124 can include an inner bore configured to receive a portion of an actuator member, such as in the form of a substantially straight threaded rod 126 (or bolt) as shown in the illustrated example. The threaded rod 126 also may be referred to herein as actuator 126, actuator member 126, and/or screw actuator 126. In examples where the delivery apparatus can be releasably connected to the outflow end 108 of the frame 102, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122. The threaded rod 126 has external threads that engage internal threads of the bore of the nut 127. The inner bore of the second post 124 (through which the threaded rod 126 extends) can have a smooth and/or non-threaded inner surface to allow the threaded rod 126 to slide freely within the bore. Rotation of the threaded rod 126 relative to the nut 127 produces radial expansion and compression of the frame 102, as further described below.

In some examples, the threaded rod 126 can extend past the nut 127 toward the inflow end 109 of the frame 102 into the inner bore of the first post 122. The nut 127 can be held in a fixed position relative to the first post 122 such that the nut 127 does not rotate relative to the first post 122. In this way, whenever the threaded rod 126 is rotated (for example, by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the first post 122 unless the threaded rod 126 is rotated relative to the nut 127. Thus, the threaded rod 126 can be retained or held by the nut 127 and can only be moved relative to the nut 127 and/or the first post 122 by rotating the threaded rod 126 relative to the nut 127 and/or the first post 122. In some examples, in lieu of using the nut 127, at least a portion of the inner bore of the first post 122 can be threaded. For example, the bore along the end portion 128 of the first post 122 can comprise inner threads that engage the external threaded rod 126 such that rotation of the threaded rod causes the threaded rod 126 to move axially relative to the first post 122.

When a threaded rod 126 extends through and/or is otherwise coupled to a pair of axially aligned posts 122, 124, the pair of axially aligned posts 122, 124 and the threaded rod 126 can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic valve 100 can include six pairs of posts 122, 124, and each of the six pairs of posts 122, 124 with their corresponding rods 126 can be configured as one of the expansion and locking mechanisms 106 for a total of six expansion and locking mechanisms 106. In some examples, not all pairs of posts 122, 124 need be expansion and locking mechanisms (that is, actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic valve 100). Specifically, when the threaded rod 126 is rotated relative to the nut 127, the first post 122, and the second post 124, the first and second posts 122, 124 can move axially relative to one another, thereby widening or narrowing the gap G (FIG. 2B) separating the posts 122, 124, and thereby radially compressing or radially expanding the prosthetic valve 100, respectively. Thus, the gap G (FIG. 2B) between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, the head portion 131 can removably or releasably couple the threaded rod 126 to a respective actuator assembly of a delivery apparatus that can be used to radially expand and/or radially compress the prosthetic valve 100 (for example, the delivery apparatus 200 of FIG. 3, as described below). Second, the head portion 131 can prevent the second post 124 from moving proximally relative to the threaded rod 126 and can apply a distally directed force to the second post 124, such as when radially expanding the prosthetic valve 100. Specifically, the head portion 131 can have a width greater than a diameter of the inner bore of the second post 124 such that the head portion 131 is prevented from moving into the inner bore of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic valve 100.

The threaded rod 126 also can include a stopper 132 (for example, in the form of a nut, washer or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (for example, clockwise) can cause corresponding axial movement of the first and second posts 122, 124 toward one another, thereby decreasing the gap G and radially expanding the frame 102, while rotation of the threaded rod 126 in an opposite second direction causes corresponding axial movement of the first and second posts 122, 124 away from one another, thereby increasing the gap G and radially compressing the frame. When the threaded rod 126 is rotated in the first direction, the head portion 131 of the rod 126 bears against an adjacent surface of the frame (for example, an outflow apex 119b), while the nut 127 and the first post 122 travel proximally along the threaded rod 126 toward the second post 124, thereby radially expanding the frame. As the frame 102 moves from a compressed configuration to an expanded configuration, the gap G between the first and second posts 122, 124 can narrow.

When the threaded rod 126 is rotated in the second direction, the threaded rod 126 and the stopper 132 move toward the outflow end 108 of the frame until the stopper 132 abuts the inflow end 170 of the second post 124 (as shown in FIGS. 2A and 2B). Upon further rotation of the rod 126 in the second direction, the stopper 132 can apply a proximally directed force to the second post 124 to radially compress the frame 102. Specifically, during crimping/radial compression of the prosthetic valve 100, the threaded rod 126 can be rotated in the second direction (for example, counterclockwise) causing the stopper 132 to push against (that is, provide a proximally directed force to) the inflow end 170 of the second post 124, thereby causing the second post 124 to move away from the first post 122, and thereby axially elongating and radially compressing the prosthetic valve 100.

Thus, each of the second posts 124 can slide axially relative to a corresponding one of the first posts 122 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic valve 100 while the stopper 132 can apply a proximally directed force to the second post 124 to radially compress the prosthetic valve 100. As explained above, radially expanding the prosthetic valve 100 axially foreshortens the prosthetic valve 100, causing an inflow end portion 134 and outflow end portion 136 of the prosthetic valve 100 (FIGS. 1A and 1B) to move towards one another axially, while radially compressing the prosthetic valve 100 axially elongates the prosthetic valve 100, causing the inflow and outflow end portions 134, 136 to move away from one another axially.

In some examples, the threaded rod 126 can be fixed against axial movement relative to the second post 124 (and the stopper 132 can be omitted) such that rotation of the threaded rod 126 in the first direction produces proximal movement of the nut 127 and radial expansion of the frame 102 and rotation of the threaded rod 126 in the second direction produces distal movement of the nut 127 and radial compression of the frame 102.

As also introduced above, some of the posts 104 can be configured as support posts 107. As shown in FIGS. 2A and 2B, the support posts 107 can extend axially between the inflow and outflow ends 109, 108 of the frame 102 and each can have an inflow end portion 138 and an outflow end portion 139. The outflow end portion 139 of one or more support posts 107 can include a commissure support structure or member 144. The commissure support structure 144 can comprise strut portions defining a commissure opening 146 therein.

The commissure opening 146 (which can also be referred to herein as a “commissure window 146”) can extend radially through a thickness of the support post 107 and can be configured to accept a portion of a valvular structure 150 (for example, a commissure 152) to couple the valvular structure 150 to the frame 102. For example, each commissure 152 can be mounted to a respective commissure support structure 144, such as by inserting a pair of commissure tabs of adjacent leaflets 158 through the commissure opening 146 and suturing the commissure tabs to each other and/or the commissure support structure 144. In some examples, the commissure opening 146 can be fully enclosed by the support post 107 such that a portion of the valvular structure 150 can be slid radially through the commissure opening 146, from an interior to an exterior of the frame 102, during assembly. In the illustrated example, the commissure opening 146 has a substantially rectangular shape that is shaped and sized to receive commissure tabs of two adjacent leaflets therethrough. However, in some examples, the commissure opening can have any of various shapes (for example, square, oval, square-oval, triangular, L-shaped, T-shaped, C-shaped, etc.).

The commissure openings 146 are spaced apart about the circumference of frame 102 (or angularly spaced apart about frame 102). The spacing may or may not be even. In one example, the commissure openings 146 are axially offset from the outflow end 108 of the frame 102 by an offset distance d₃ (indicated in FIG. 2A). As an example, the offset distance d₃ may be in a range from 2 mm to 6 mm. In general, the offset distance d₃ should be selected such that when the leaflets are attached to the frame 102 via the commissure openings 146, the free edge portions (for example, outflow edge portions) of the leaflets 158 will not protrude from or past the outflow end 108 of the frame 102.

The frame 102 can comprise any number of support posts 107, any number of which can be configured as commissure support structures 144. For example, the frame 102 can comprise six support posts 107, three of which are configured as commissure support structures 144. However, in some examples, the frame 102 can comprise more or less than six support posts 107 and/or more or less than three commissure support structures 144.

The inflow end portion 138 of each support post 107 can comprise an extension 154 (show as a cantilevered strut in FIGS. 2A and 2B) that extends toward the inflow end 109 of the frame 102. Each extension 154 can comprise an aperture 156 extending radially through a thickness of the extension 154. In some examples, the extension 154 can extend such that an inflow edge of the extension 154 aligns with or substantially aligns with the inflow end 109 of the frame 102. In use, the extension 154 can prevent or mitigate portions of an outer skirt from extending radially inwardly and thereby prevent or mitigate any obstruction of flow through the frame 102 caused by the outer skirt. The extensions 154 can further serve as supports to which portions of the inner and/or outer skirts and/or the leaflets and/or the connecting skirt 125 can be coupled. For example, sutures used to connect the inner and/or outer skirts and/or the leaflets and/or the connecting skirt 125 can be wrapped around the extensions 154 and/or can extend through apertures 156.

As an example, each extension 154 can have an aperture 156 (FIG. 2A) or other features to receive a suture or other attachment material for connecting an adjacent inflow edge portion 160 of a leaflet 158 (FIG. 1A), the outer skirt 103 (in FIG. 1B), the connecting skirt 125, and/or an inner skirt. In some examples, the inflow edge portion 160 of each leaflet 158 can be connected to a corresponding extension via a suture 135 (FIG. 1A).

In some examples, the outer skirt 103 can be mounted around the outer surface of frame 102 as shown in FIG. 1B and the inflow edge of the outer skirt 103 (lower edge in FIG. 1B) can be attached to the connecting skirt 125 and/or the inflow edge portions 160 of the leaflets 158 that have already been secured to frame 102 as well as to the extensions 154 of the frame by sutures 129. The outflow edge of the outer skirt 103 (the upper edge in FIG. 1B) can be attached to selected struts with stitches 137. In implementations where the prosthetic valve includes an inner skirt, the inflow edge of the inner skirt can be secured to the inflow edge portions 160 before securing the cusp edge portions to the frame so that the inner skirt will be between the leaflets and the inner surface of the frame. After the inner skirt and leaflets are secured in place, then the outer skirt can be mounted around the frame as described above.

The frame 102 can be a unitary and/or fastener-free frame that can be constructed from a single piece of material (for example, Nitinol, stainless steel or a cobalt-chromium alloy), such as in the form of a tube. The plurality of cells can be formed by removing portions (for example, via laser cutting) of the single piece of material. The threaded rods 126 can be separately formed and then be inserted through the bores in the second (proximal) posts 124 and threaded into the threaded nuts 127.

In some examples, the frame 102 can include a plastically-expandable material, such as stainless steel or a cobalt-chromium alloy. When the frame includes a plastically-expandable material, the prosthetic valve 100 can be placed in a radially compressed state along the distal end portion of a delivery apparatus for insertion into a patient's body. When at the desired implantation site, the frame 102 (and therefore the prosthetic valve 100) can be radially expanded from the radially compressed state to a radially expanded state via actuation of actuator assemblies of the delivery apparatus (as further described below), which rotate the rods 126 to produce expansion of the frame 102. During delivery to the implantation site, the prosthetic valve 100 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

In some examples, the frame 102 can include a self-expandable material (for example, Nitinol). When the frame 102 includes a self-expandable material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule. In some examples, the frame (and therefore the prosthetic valve) can partially self-expand from the radially compressed state to a partially radially expanded state. The frame 102 (and therefore the prosthetic valve 100) can be further radially expanded from the partially expanded state to a further radially expanded state via actuation of actuator assemblies of the delivery apparatus (as further described below), which rotate the rods 126 to produce expansion of the frame.

As introduced above, the threaded rods 126 can removably couple the prosthetic valve 100 to actuator assemblies of a delivery apparatus. Referring to FIG. 3, it illustrates an example of a delivery apparatus 200 for delivering a prosthetic device or valve 202 (for example, prosthetic valve 100) to a desired implantation location. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 204, a first elongated shaft 206 (which comprises an outer shaft in the illustrated example) extending distally from the handle 204, at least one actuator assembly 208 extending distally through the first shaft 206, a second elongated shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 202 when actuated, such as by one or more knobs 211, 212, 214 included on the handle 204 of the delivery apparatus 200.

Though the illustrated example shows two actuator assemblies 208 for purposes of illustration, it should be understood that one actuator assembly 208 can be provided for each actuator (for example, actuator or threaded rod 126) on the prosthetic valve. For example, three actuator assemblies 208 can be provided for a prosthetic valve having three actuators. In some examples, a greater or fewer number of actuator assemblies can be present.

In some examples, a distal end portion 218 of the shaft 206 can be sized to house the prosthetic valve in its radially compressed, delivery state during delivery of the prosthetic valve through the patient's vasculature. In this manner, the distal end portion 218 functions as a delivery sheath or capsule for the prosthetic valve during delivery,

The actuator assemblies 208 can be releasably coupled to the prosthetic valve 202. For example, in the illustrated example, each actuator assembly 208 can be coupled to a respective actuator (for example, threaded rod 126) of the prosthetic valve 202. Each actuator assembly 208 can comprise a support tube and an actuator member. When actuated, the actuator assembly can transmit pushing and/or pulling forces to portions of the prosthetic valve to radially expand and collapse the prosthetic valve as previously described. The actuator assemblies 208 can be at least partially disposed radially within, and extend axially through, one or more lumens of the first shaft 206. For example, the actuator assemblies 208 can extend through a central lumen of the shaft 206 or through separate respective lumens formed in the shaft 206.

The handle 204 of the delivery apparatus 200 can include one or more control mechanisms (for example, knobs, buttons, or other actuating mechanisms) for controlling different components of the delivery apparatus 200 in order to expand and/or deploy the prosthetic valve 202. For example, in the illustrated example, the handle 204 comprises first, second, and third knobs 211, 212, and 214, respectively and at least one switch, such as in the form of a button 213. The handle 204 of the delivery apparatus 200 can also include one or more indicators (for example, visual indicators such as lights and/or displays, auditory indicators, etc.) that provide feedback to the user on the status of certain components of the delivery apparatus and/or the prosthetic valve, as further described below. For example, in the illustrated example, the handle 204 comprises first and second visual indicators 215 and 216, respectively, which can be light-emitting elements, such as LED's.

The first knob 211 can be a rotatable knob configured to produce axial movement of the first shaft 206 relative to the prosthetic valve 202 in the distal and/or proximal directions in order to deploy the prosthetic valve from the delivery sheath 218 once the prosthetic valve has been advanced to a location at or adjacent the desired implantation location with the patient's body. For example, rotation of the first knob 211 in a first direction (for example, clockwise) can retract the sheath 218 proximally relative to the prosthetic valve 202 and rotation of the first knob 211 in a second direction (for example, counter-clockwise) can advance the sheath 218 distally. In some examples, the first knob 211 can be actuated by sliding or moving the first knob 211 axially, such as pulling and/or pushing the knob. In some examples, actuation of the first knob 211 (rotation or sliding movement of the first knob 211) can produce axial movement of the actuator assemblies 208 (and therefore the prosthetic valve 202) relative to the delivery sheath 218 to advance the prosthetic valve distally from the sheath 218.

The second knob 212 can be a rotatable knob configured to produce radial expansion and/or compression of the prosthetic valve 202. For example, rotation of the second knob 212 can rotate the threaded rods of the prosthetic valve 202 via the actuator assemblies 208. Rotation of the second knob 212 in a first direction (for example, clockwise) can radially expand the prosthetic valve 202 and rotation of the second knob 212 in a second direction (for example, counter-clockwise) can radially collapse the prosthetic valve 202. In some examples, the second knob 212 can be actuated by sliding or moving the second knob 212 axially, such as pulling and/or pushing the knob.

The button 213 can be configured to magnetically couple the actuator assemblies 208 to the prosthetic valve 202 and to magnetically de-couple the actuator assemblies 208 from the prosthetic valve. Specifically, pressing the button 213 can magnetize and/or demagnetize one or more components of the actuator assemblies 208. For example, the button 213 can control a supply of an electrical current to an electromagnet included in an actuator assembly 208. Pressing the button 213 a single time, for example, can magnetize the actuator assembly 208 and pressing the button 213 a second time, for example, can demagnetize the actuator assembly 208.

In some examples, each actuator assembly 208 can have a respective electromagnet and the button can be operable to magnetize or de-magnetize all of the electromagnets at the same time with a single operation of the button. In this way, the button 213 can enable magnetic coupling of the actuator assemblies 208 to the threaded rods 126 (for example, during radial expansion and/or compression of the prosthetic valve 202). The button 213 can be used to magnetically uncouple the actuator assemblies 208 and the threaded rods 126 prior to disconnecting the actuator assemblies 208 from the proximal portions of the actuators of the prosthetic valve (for example, the threaded rods). In some examples, the handle 204 can comprise two buttons instead of a single button 213. For example, the handle 204 can comprise a first button to magnetize one or more components of the actuator assemblies 208 and a second button to demagnetize the components of the actuator assemblies 208. In some examples, the button or buttons 213 can comprise one or more different input devices such as knobs, switches, toggles or the like.

The third knob 214 can be a rotatable knob operatively connected to a proximal end portion of each actuator assembly 208. The third knob 214 can be configured to retract an outer sleeve or support tube of each actuator assembly 208 away from the actuators of the prosthetic valve. In some examples of the delivery apparatus, as further described, the outer sleeves can be retracted by simply retracting the entire delivery device after demagnetizing the connection between the actuator assemblies and the prosthetic valve, in which case the third knob 214 can be omitted. Once the actuator assemblies 208 are uncoupled from the prosthetic valve 202, the delivery apparatus 200 can be removed from the patient, leaving just the prosthetic valve 202 in the patient.

The first and second visual indicators 215, 216 can be LED indicators configured to indicate whether the actuator assemblies 208 are coupled to, connected to, and/or engaged with the actuators 126 of the prosthetic valve 100. For example, the first visual indicator 215 can be a first color (for example, green) and can be illuminated to indicate when the actuator assemblies 208 are coupled to, connected to, and/or engaged with the actuators 126 (for example, an engaged state). The second visual indicator 216 can be a second color (for example, red) and can be illuminated to indicate when the actuator assemblies 208 are decoupled from, not connected to, and/or disengaged from the actuators 126 (for example, a disengaged state).

In some examples, the first and second visual indicators 215, 216 (or additional indicators, such as third and fourth visual indicators (not shown)) can be LED indicators configured to indicate whether the actuator assemblies 208 are magnetized or demagnetized. For example, the first visual indicator 215 can be a first color (for example, green) and can be illuminated to indicate when the actuator assemblies 208 are magnetized (for example, after pressing the button 213 a first time). The second visual indicator 216 can be a second color (for example, red) and can be illuminated to indicate when the actuator assemblies 208 are demagnetized (for example, after pressing the button 213 a second time).

As introduced above, an actuator assembly, such as any actuator assembly described herein, of a delivery apparatus can be coupled to the head portion 131 of each threaded rod 126. For example, when coupled together, the actuator assembly and the threaded rod 126 are in an engaged state. In the engaged state, rotation of one or more components of the actuator assembly (e.g., a driver of the actuator assembly, etc.) can result in rotation of the threaded rod 126. Specifically, in the engaged state, at least one surface of the actuator assembly (e.g., at least one surface of the driver) contacts at least one surface of the threaded rod 126. When the actuator assembly and the threaded rod 126 are not coupled together, the actuator assembly and the threaded rod 126 are in a disengaged state. In the disengaged state, rotation of one component (e.g., the driver of the actuator assembly) does not result in rotation of the other component (e.g., the threaded rod 126). Specifically, in the disengaged state, at least the driver of the actuator assembly does not contact the threaded rod 126. In some examples, in the disengaged state, no portion of the actuator assembly contacts the threaded rod 126.

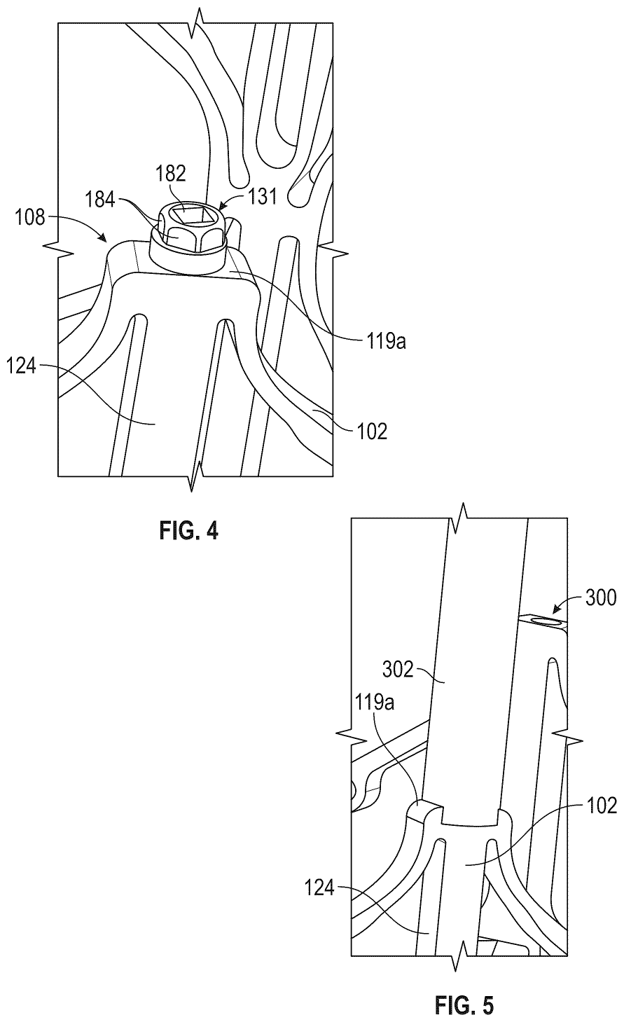

As shown in FIGS. 4 and 8, the head portion 131 can be included at a proximal end portion 180 of the threaded rod 126 and can extend proximally past a proximal end of a second post 124. The head portion 131 can comprise a central bore 182 and in some examples, as depicted, one or more facets 184. In some examples, as depicted, the central bore 182 can comprise a non-circular bore, such as a square bore. In some examples, the central bore 182 can comprise a circular bore (see FIG. 12). The facets 184 can be disposed on an exterior surface of the head portion 131. In some examples, as depicted, the head portion 131 comprises six facets, each positioned at 60° on the exterior surface relative to others of the facets. In some examples, the head portion 131 can comprise a substantially smooth, continuously curved exterior surface.

As discussed above, the head portion 131 can have a width greater than a diameter of the inner bore of the second post 124 such that the head portion 131 is prevented from moving into the inner bore of the second post 124 and such that the head portion 131 abuts the outflow end 108 of the frame 102. In particular, the head portion 131 can abut an outflow apex 119b of the frame 102. The head portion 131 can be used to apply a distally-directed force to the second post 124, for example, during radial expansion of the frame 102.

Referring to FIGS. 5-9, each actuator assembly 300 can comprise a first actuation member configured as a support tube or outer sleeve 302 and a second actuation member configured as a driver 304. The driver 304 can extend through the outer sleeve 302. The distal end portions of the outer sleeve 302 and driver 304 can be configured to engage or abut the proximal end of the threaded rod 126 (for example, the head portion 131) and/or the frame 102 (for example, the apex 119b). The proximal portions of the outer sleeve 302 and driver 304 can be operatively coupled to the handle of a delivery apparatus (for example, handle 204). In some instances, the outer sleeve 302 can be optional, such that the actuator assembly 300 only includes one actuation member, the driver 304. The delivery apparatus in this example can include the same features described previously for delivery apparatus 200. In some examples, the proximal end portions of each driver 304 can be operatively connected to the knob 212 such that rotation of the knob 212 (clockwise or counterclockwise) causes corresponding rotation of the drivers 304. The proximal end portions of each outer sleeve 302 can be operatively connected to the knob 214 such that rotation of the knob 214 (clockwise or counterclockwise) causes corresponding axial movement of the sleeves 302 (proximally or distally) relative to the drivers 304. In some examples, the handle can include electric motors for actuating these components.

As shown in FIG. 7, the driver 304 can comprise a driver head portion 306 at the distal end portion of the driver 304. The driver head portion 306 can be configured to extend into the bore 182 of the head portion 131 of the threaded rod 126. In some examples, the driver head portion 306 can comprise one or more facets 308 that correspond to the shape of the central bore 182. In some examples, as depicted, the driver head portion 306 comprises four facets 308 with rounded edges between the facets 308. The driver head portion 306 can be configured to magnetically couple the driver 304 to the threaded rod 126. For example, the driver head portion 306 can comprise an electromagnet, such that the driver head portion 306 (also referred to herein as electromagnet 306) can selectively induce a magnetic field. The magnetic field induced by the electromagnet 306 can be sufficient to retain engagement between the driver 304 and the threaded rod 126 during delivery and radial expansion of the prosthetic valve 202. In particular, the electromagnet 306, when energized, can resist axial separation of the driver 304 from the threaded rod 126. In some examples, the entire driver 304 can comprise the electromagnet. Additionally or alternatively, the outer sleeve 302 can comprise an electromagnet in some examples.

One or more wires 310 can be electrically coupled to the driver head portion 306 to provide electric power to the electromagnet, as shown in FIG. 9. The wires 310 can extend through the driver 304 to the handle 204 and be electrically coupled to the button 213 and/or the indicators 215, 216 (see also FIG. 3). For example, the wires 310 can extend through a lumen of a proximal shaft of the driver 304, thereby isolating the wires 310 from the surrounding environment. In some examples, the wires 310 can extend through a lumen of the outer sleeve 302.

The electromagnet can comprise a wire coil 318 wound around a ferromagnetic core. For example, all or a portion of the driver head portion 306 can made of a ferromagnetic material, such as iron, cobalt, nickel, or alloys thereof. The wire coil 318 can be wound around the ferromagnetic portion of the driver head portion 306. Distal ends of the wires 310 can be in electrical contact with the wire coil 318. The head portion 131 of the rod 126, or the entire rod 126, similarly can be made of a ferromagnetic material, such as iron, cobalt, nickel, or alloys thereof, so as to be magnetically attracted to the electromagnet when it is energized.

As shown in FIG. 9, to couple the actuator assembly 300 to the threaded rod 126, the driver 304 can be positioned in an engaged state with the threaded rod 126, such that a distal end of the driver head portion 306 is disposed within the bore 182 and contacts a surface thereof. The driver head portion 306 can induce a magnetic field (for example, using button 213 on the handle 204 of the delivery apparatus 200) to place the actuator assembly 300 in a magnetized or magnetic state. In the magnetic state, the magnetic field induced by driver head portion 306 can supply an attractive magnetic force to help facilitate and/or maintain the engaged state between the driver 304 to the threaded rod 126.

In some examples, the driver head portion 306 can be placed in the magnetic state when the actuator assembly 300 and the threaded rod 126 are in a disengaged state, such that the driver head portion 306 is in the magnetic state prior to the driver head portion 306 being positioned within the bore 182 of the threaded rod 126. In some examples, the driver head portion 306 can be in a demagnetized or nonmagnetic state when the actuator assembly 300 and the rod 126 are in the engaged state, such that the driver head portion 306 is in a nonmagnetic state when the driver head portion 306 is positioned within the bore 182 and contacts a surface thereof. Subsequently, the driver head portion 306 can be magnetized and placed in the magnetic state while the actuator assembly 300 and the rod 126 are in the engaged state (e.g., after the driver head portion 306 is positioned within the bore 182). In some examples, the magnetic state can assist with positioning and/or alignment of the driver head portion 306 within the bore 182, thereby simplifying engagement of the driver 304 with the threaded rod 126.

Because the driver head portion 306 of the driver 304 extends into the bore 182 of the threaded rod 126 when the driver 304 and the threaded rod 126 are coupled in the engaged state, the driver 304 and the threaded rod 126 can be axially locked relative to each other. The engagement of the facets 308 with those of the bore 182 of the head portion 131 prevents relative rotation of the driver 304 and the threaded rod 126. So coupled, the driver 304 can be rotated (for example, using knob 212 on the handle 204 of the delivery apparatus 200) to cause corresponding rotation of the threaded rod 126 to radially expand or radially compress the prosthetic device. The driver 304 can be in a magnetized state during radial expansion or compression of the prosthetic device. The magnetic field induced by the electromagnet 306 can be sufficient to retain the engagement of the driver 304 with the threaded rod 126 during such rotation. In this way, the magnetic state of the electromagnet 306 can facilitate and/or maintain the engaged state of the driver 304 and the threaded rod 126. The driver head portion 306 can be configured (for example, sized and shaped) such that it is advantageously spaced apart from the inner walls of the outer sleeve 302, such that the driver head portion 306 does not frictionally contact the outer sleeve 302 during rotation.

Though in the illustrated example the driver head portion 306 has a substantially square shape in cross-section, in some examples, the driver head portion 306 can have any of various shapes, for example, square, triangular, oval, etc. The bore 182 can be correspondingly shaped to receive the driver head portion 306. In some examples, the magnetic force alone can be sufficient to prevent relative rotation between the driver 304 and the threaded rod 126 (in which case one or both of the driver head portion 306 and the bore 182 can have circular cross-sectional profiles).

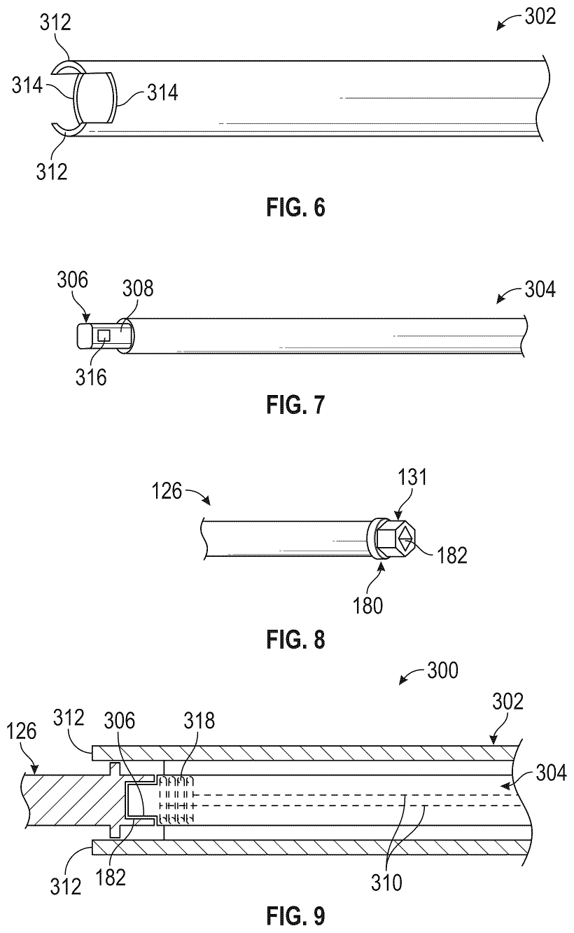

The outer sleeve 302 can be advanced distally relative to the driver 304 past the driver head portion 306, until the outer sleeve 302 engages the frame 102 (for example, a second post 124 of the frame 102). As shown in FIG. 6, the distal end portion of the outer sleeve 302 also can comprise first and second support extensions 312 defining gaps or notches 314 between the extensions 312. The support extensions 312 can be oriented such that, when the actuator assembly 300 is coupled to a respective threaded rod 126, the support extensions 312 extend partially over an adjacent end portion (for example, the upper end portion) of one of the posts 124, on opposite sides of the post 124. The engagement of the support extensions 312 with the frame 102 in this manner can counter-act rotational forces applied to the frame 102 by the rods 126 during expansion of the frame 102. In the absence of a counter-force acting against these rotational forces, the frame can tend to “jerk” or rock in the direction of rotation of the rods when they are actuated to expand the frame. The illustrated configuration is advantageous in that outer sleeves, when engaging the proximal posts 124 of the frame 102, can prevent or mitigate such jerking or rocking motion of the frame 102 when the frame 102 is radially expanded.

Each actuator assembly 300 can include one or more sensors 316 configured to detect resistance of the threaded rods 126 to rotation during radial expansion and radial compression of the prosthetic valve 202. For example, a sensor 316 can be disposed on an outer surface of the driver head portion 306 to detect the resistive forces exerted by the threaded rods 126 onto the drivers 304. In this way, the head portion 131 of the threaded rod 126 can contact the sensor 316 when the driver head portion 306 is positioned within the bore 182 and the threaded rod 126 is turned by the driver 304. In some examples, the sensors 316 can detect whether the actuator assembly 300 is in an engaged state with the threaded rods 126. In some examples, the sensors 316 can be positioned on other components of the actuator assembly 300 to detect the resistive forces exerted by the threaded rods 126 and/or the forces exerted by the drivers 304 to radially expand the prosthetic heart valve 202.

In some examples, the resistive forces detected by the sensors 316 can provide an indication of the curvature or overall shape of the native annulus or lumen in which the prosthetic valve is being implanted. For example, anatomical regions with greater curvature may result in greater resistance of the threaded rods 126 to rotation. This may be interpreted to provide an indication of the degree of curvature (that is, non-circularity) at the site of implantation. In some examples, the curvature of the implantation site may be indicated on a display on a handle of the delivery apparatus (for example, an LCD display on handle 204 of delivery apparatus 200). In some examples, the sensors 316 may wirelessly transmit the indication of the curvature to a remote device.

To decouple the actuator assembly 300 from the prosthetic valve 202, the driver 304 can be demagnetized to allow the sleeve 302 and/or the driver 304 to be withdrawn proximally relative to the threaded rod 126. Specifically, when the driver 304 is in a demagnetized state, the driver 304 can be transitioned from the engaged state to the disengaged state, such that the driver 304 can be released from the bore 182 of the threaded rod 126, thereby decoupling the driver 304 from the threaded rod 126.

The sleeve 302 can be advanced (moved distally) and/or retracted (moved proximally) relative to the driver 304 via a control mechanism (for example, knob 214) on the handle 204 of the delivery apparatus 200, by an electric motor, and/or by another suitable actuation mechanism. For example, the physician can turn the knob 214 in a first direction to move the sleeve 302 distally relative to the driver 304 and the prosthetic valve and can turn the knob 214 in an opposite second direction to move the sleeve 302 proximally relative to the driver 304 and the valve. Thus, when the sleeve 302 does not abut the prosthetic device and the physician rotates the knob 214 in the first direction, the sleeve 302 can move distally relative to the driver 304, thereby advancing the sleeve 302 over the driver 304. Further, when the physician rotates the knob 214 in the second direction the sleeve 302 can move proximally relative to the driver 304, thereby withdrawing/retracting the sleeve 302 from the driver 304.

In some examples, the actuator assembly 300 can be coupled to or removed from the prosthetic valve 202 without need to advance or retract the sleeve 302 relative to the driver 304. In such examples, the sleeve 302 need not be movable in the distal and proximal directions relative to the driver 304 and the knob 214 can be omitted. Instead, the sleeve 302 can be advanced over and retracted from the apex 119a by advancing the delivery apparatus toward and away from the apex 119a.

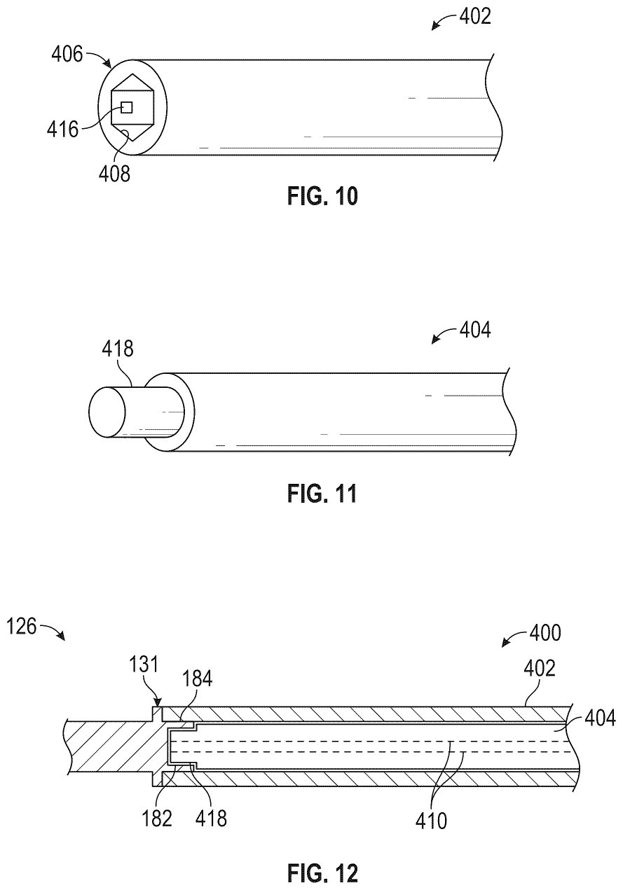

FIGS. 10-12 illustrate an example of an actuator assembly 400. Each actuator assembly 400 can comprise a first actuation member configured as a driver 402 (FIG. 10) and a second actuation member configured as an inner support member 404 (FIG. 11). The inner support member 404 can extend through the outer driver 402. The distal end portions of the driver 402 and support member 404 can be configured to engage or abut the proximal end of the threaded rod 126 (for example, the head portion 131) and/or the frame 102 (for example, the apex 119b). The proximal portions of the driver 402 and support member 404 can be operatively coupled to the handle of a delivery apparatus (for example, handle 204).

The delivery apparatus in this example can include the same features described previously for delivery apparatus 200. In some examples, the proximal end portions of each driver 402 can be operatively connected to the knob 212 such that rotation of the knob 212 (clockwise or counterclockwise) causes corresponding rotation of the drivers 402. The proximal end portions of each support member 404 can be operatively connected to the knob 214 such that rotation of the knob 214 (clockwise or counterclockwise) causes corresponding axial movement of the support members 404 (proximally or distally) relative to the drivers 402. In some examples, the handle can include electric motors for actuating these components.

As shown in FIG. 10, the driver 402 can comprise a driver head portion 406 at the distal end of the driver 402. The driver head portion 406 can be configured to extend over the head portion 131 of the threaded rod 126 and engage with the external facets 184. In some examples, the driver head portion 406 can comprise an opening or socket 408 that corresponds to the shape of the head portion 131 to transfer rotation of the driver 402 to the rod 126. For example, as depicted, the driver head portion 406 comprises a hex-shaped opening 408 with internal facets that can engage the external facets 184 of the rod 126.

The inner support member 404 can be configured to magnetically couple the inner support member 404 (and the actuator assembly 400) to the threaded rod 126. For example, a distal head portion 418 of the inner support member 404 can comprise an electromagnet, such that the distal head portion 418 (also referred to herein as electromagnet 418) can selectively induce a magnetic field. The magnetic field induced by the electromagnet 418 can be sufficient to retain engagement between the driver 402 and the threaded rod 126 during delivery and radial expansion of the prosthetic valve 202.

In some examples, the driver 402 can comprise the electromagnet. In these examples, the support member 404 can be optional, such that the actuator assembly 400 only includes one actuation member, namely, the driver 402 that comprises the electromagnet. In some examples, both the driver 402 and the support member 404 can comprise electromagnets. The electromagnets, whether incorporated in the driver 402 or the support member 404, can be configured as previously described for the example of FIGS. 6-9, and can include a ferromagnetic core and a wire coil.

The distal head portion 418 of the support member 404 can correspond to the shape of the bore 182 of the head portion 131 of the threaded rod 126. In some examples, as depicted, the distal head portion 418 of the support member 404 has a substantially smooth, continuously curved exterior surface, which can correspond to a circular bore 182.

One or more wires 410 can be electrically coupled to the inner support member 404 to provide electric power to the electromagnet, as shown in FIG. 12. The wires 410 can extend through the support member 404 to the handle 204 and be electrically operatively coupled to the button 213 and/or the indicators 215, 216. For example, the wires 410 can extend through a lumen of a proximal shaft of the support member 404, thereby isolating the wires 410 from the surrounding environment. In some examples, the wires 410 can extend through a lumen of the driver 402. If the driver 402 comprises an electromagnet, additional wires 410 can be provided to electrically couple the electromagnet of the driver 402 to the button 213 and/or the indicators 215, 216.

As shown in FIG. 12, to couple the actuator assembly 400 to the threaded rod 126, the driver 402 can be positioned in an engaged state with the threaded rod 126, such that at least a segment of the head portion 131 (for example, the segment of the head portion 131 including the facets 184) is disposed within the opening 408 of the driver head portion 406. Specifically, the driver 402 can be advanced distally relative to the threaded rod 126 past the facets 184, until the driver head portion 406 engages a flange of the head portion 131. Additionally, in the engaged state, the inner support member 404 can be positioned such that the electromagnet 418 is disposed within the bore 182.

The electromagnet 418 can induce a magnetic field (for example, using button 213 on the handle 204 of the delivery apparatus 200) to place the actuator assembly 400 in a magnetized or magnetic state. In the magnetic state, the magnetic field induced by the electromagnet 418 can supply an attractive magnetic force to help facilitate and/or maintain the engaged state between the driver 402 (and therefore, the actuator assembly 400) and the threaded rod 126. In some examples, the electromagnet 418 can be placed in a magnetized or magnetic state when the actuator assembly 400 and the threaded rod 126 are in a disengaged state, such that the electromagnet 418 is in the magnetic state prior to the electromagnet 418 being positioned within the bore 182 and/or prior to the driver head portion 406 being positioned around the head portion 131. In some examples, the electromagnet 418 can be in a demagnetized or nonmagnetic state during positioning of the actuator assembly 400 relative to the threaded rod 126 and subsequently magnetized after the actuator assembly 400 and the threaded rod 126 are in the engaged state (e.g., after the electromagnet 418 is positioned within the bore 182 and the driver head portion 406 is positioned around the head portion 131). In some examples, the magnetic state can assist with positioning and/or alignment of the driver 402 and support member 404 relative to the threaded rod 126, thereby simplifying engagement of the actuator assembly 400 with the threaded rod 126.

Because the head portion 131 of the threaded rod 126 extends into the opening 408 of the driver head portion 406 of the driver 402 when the driver 402 and the threaded rod 126 are coupled in the engaged state, the driver 402 and the threaded rod 126 can be rotationally locked such that they co-rotate. So coupled, the driver 402 can be rotated (for example, using knob 212 on the handle 204 of the delivery apparatus 200) to cause corresponding rotation of the threaded rod 126 to radially expand or radially compress the prosthetic device. The support member 404 can be in a magnetized state during delivery and radial expansion or compression of the prosthetic device. The magnetic field induced by the electromagnet 418 can be sufficient to retain the engagement of the driver 402 with the threaded rod 126 during such rotation. In this way, the magnetic state of the electromagnet 306 can facilitate and/or maintain the engaged state of the driver 304 and the threaded rod 126. The inner support member 404 can be configured (for example, sized and shaped) such that it is advantageously spaced apart from the inner walls of the driver 402, such that the driver 402 does not frictionally contact the support member 404 during rotation. Though in the illustrated example the opening 408 has a substantially hexagon shape in cross-section, in some examples, the opening 408 can have any of various shapes, for example, square, triangular, oval, etc. These shapes can correspond to a shape of the head portion 131 of the threaded rod 126.

Each actuator assembly 400 can include one or more sensors 416 configured to detect resistance of the threaded rods 126 to rotation during radial expansion and radial compression of the prosthetic valve 202. For example, a sensor 416 can be disposed on an inner surface of the driver head portion 406 to detect the resistive forces exerted by the threaded rods 126 onto the drivers 402. In this way, the head portion 131 of the threaded rod 126 can contact the sensor 416 when the head portion 131 is positioned within the driver head portion 406 and the threaded rod 126 is turned by the driver 402. In some examples, the sensors 416 can detect whether the actuator assembly 400 is in an engaged state with the threaded rods 126. In some examples, the sensors 416 can be positioned on other components of the actuator assembly 400 to detect the resistive forces exerted by the threaded rods 126 and/or the forces exerted by the drivers 402 to radially expand the prosthetic heart valve 202.

In some examples, the resistive forces detected by the sensors 416 can provide an indication of the curvature of the implantation location. For example, anatomical regions with greater curvature may result in greater resistance of the threaded rods 126 to rotation. This may be interpreted to provide an indication of the degree of curvature (that is, non-circularity) at the site of implantation. In some examples, the curvature of the implantation site may be indicated on a display on a handle of the delivery apparatus (for example, an LCD display on handle 204 of delivery apparatus 200). In some examples, the sensors 416 may wirelessly transmit the indication of the curvature to a remote device.

To decouple the actuator assembly 400 from the prosthetic valve 202, the inner support member 404 can be demagnetized to allow the driver 402 and/or the support member 404 to be withdrawn proximally relative to the threaded rod 126. Specifically, when the support member 404 is in a demagnetized state, the actuator assembly 400 can be transitioned from the engaged state to the disengaged state, such that the support member 404 can be released from the bore 182 of the threaded rod 126 and the driver head portion 406 can be released from the head portion 131, thereby decoupling the actuator assembly 400 from the threaded rod 126.

The outer driver 402 can be advanced (moved distally) and/or retracted (moved proximally) relative to the support member 404 via a control mechanism (for example, knob 214) on the handle 204 of the delivery apparatus 200, by an electric motor, and/or by another suitable actuation mechanism. For example, the physician can turn the knob 214 in a first direction to apply a distally directed force to the driver 402 and can turn the knob 214 in an opposite second direction to apply a proximally directed force to the driver 402. Thus, when the driver 402 does not abut the prosthetic device and the physician rotates the knob 214 in the first direction, the driver 402 can move distally relative to the support member 404, thereby advancing the driver 402 over the support member 404. When the physician rotates the knob 214 in the second direction the driver 402 can move proximally relative to the support member 404, thereby withdrawing/retracting the driver 402 from the support member 404.

In some examples, the actuator assembly 400 can be coupled to and removed from the prosthetic valve 202 without need to advance or retract the driver 402 relative to the support member 404. In such examples, the driver 402 need not be movable in the distal and proximal directions relative to the support member 404 and the knob 214 can be omitted. Instead, the driver 402 can be advanced over and retracted from the rod 126 by advancing the delivery apparatus toward and away from the rod 126.

In some examples, one or more electromagnets of a delivery apparatus can be used to magnetically coupled a component of a delivery apparatus to an actuator of a prosthetic valve, wherein the actuator of the prosthetic valve is a linear actuator configured to produce radial compression or expansion of the prosthetic valve. Examples of prosthetic valves having one or more linear actuators (also referred to as push-pull actuators) are disclosed on U.S. Publication No. 2018/0153689, which is incorporated herein by reference. A linear actuator can have an outer member and an inner member coupled to axially spaced locations on the frame of the prosthetic valve. The outer and inner members can reciprocate or slide relative to each other to produce radial expansion and compression of the frame. The delivery apparatus can have one or more actuator assemblies configured to be releasably coupled to respective linear actuators of the prosthetic valve and transfer pushing and pulling forces to the linear actuators of the prosthetic valve. Each actuator assembly can have an electromagnet as disclosed herein for forming a releasable connection with a linear actuator of the prosthetic valve. For example, an actuator assembly of the delivery apparatus can have outer and inner members configured to be releasably coupled to respective outer and inner members of the prosthetic valve. One or both of the outer and inner members of the actuator assembly can include an electromagnet configured to form a magnetic coupling with respective outer and inner members of the prosthetic valve.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (e.g., with heat, radiation, and/or chemicals, etc.) to ensure they are safe for use with patients, and any of the methods herein can include sterilization of the associated system, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus for implanting a prosthetic valve comprising a mechanically actuatable frame comprising at least one actuator member, the actuator member configured to be rotated to radially expand or compress the frame, the delivery apparatus comprising: at least one actuator assembly configured to actuate the actuator member of the prosthetic valve, the actuator assembly comprising an outer sleeve member having a lumen and an inner driver member coaxially disposed within the lumen of the sleeve member, the driver member being rotatable relative to the sleeve member, wherein the actuator assembly is configured to be transitioned between an engaged state and a disengaged state with the prosthetic valve, and wherein the driver member is configured to be selectively magnetically coupled to the actuator member in the engaged state.

Example 2. The delivery apparatus of any example herein, particularly example 1, wherein the driver member includes an electromagnet configured to induce an electromagnetic field.

Example 3. The delivery apparatus of any example herein, particularly example 2, further comprising a handle coupled to the at least one actuator assembly.

Example 4. The delivery apparatus of any example herein, particularly example 3, wherein the handle comprises an input device operatively coupled to the driver member, wherein the input device is configured to magnetize and demagnetize the electromagnet.

Example 5. The delivery apparatus of any example herein, particularly example 4, further comprising one or more wires electrically operatively coupled to the electromagnet and the input device, wherein the one or more wires extend through a lumen of the driver member.

Example 6. The delivery apparatus of any example herein, particularly either example 4 or example 5, wherein the handle comprises one or more visual indicators to indicate whether the electromagnet is magnetized or demagnetized and/or whether the at least one actuator assembly is in the engaged state or the disengaged state.

Example 7. The delivery apparatus of any example herein, particularly any one of examples 1-6, wherein the delivery apparatus comprises a plurality of actuator assemblies.

Example 8. The delivery apparatus of any example herein, particularly example 7, wherein each actuator assembly further comprises a sensor configured to detect a resistive force exerted by the actuator member on the driver member to resist rotation.

Example 9. The delivery apparatus of any example herein, particularly example 8, wherein the delivery apparatus further comprises an indicator configured to indicate a curvature of an implantation site of the prosthetic valve based on the resistive forces detected by the sensors.

Example 10. A delivery apparatus for a prosthetic valve, the delivery apparatus comprising: a handle; and a driver having a proximal end portion coupled to the handle and a distal end portion comprising an electromagnet configured to releasably and magnetically couple the driver to an actuator of the prosthetic valve.

Example 11. The delivery apparatus of any example herein, particularly example 10, further comprising a sleeve member coupled to the handle, wherein the driver extends through the sleeve member.

Example 12. The delivery apparatus of any example herein, particularly either example 10 or example 11, wherein the handle comprises an input device operatively coupled to the driver, wherein the input device is configured to magnetize and demagnetize the electromagnet.

Example 13. The delivery apparatus of any example herein, particularly example 12, further comprising one or more wires electrically operatively coupled to the electromagnet and the input device, wherein the one or more wires extend through a lumen of the driver.

Example 14. The delivery apparatus of any example herein, particularly any one of examples 10-13, wherein the handle comprises one or more visual indicators to indicate whether the electromagnet is magnetized or demagnetized and/or whether the driver is in an engaged state or a disengaged state with the actuator.

Example 15. The delivery apparatus of any example herein, particularly any one of examples 10-14, wherein the delivery apparatus comprises a plurality of drivers.

Example 16. The delivery apparatus of any example herein, particularly example 15, wherein each driver further comprises a sensor configured to detect a resistive force exerted by the actuator on the driver to resist rotation.

Example 17. The delivery apparatus of any example herein, particularly example 16, wherein the delivery apparatus further comprises an indicator configured to indicate a curvature of an implantation site of the prosthetic valve based on the resistive forces detected by the sensors.

Example 18. An assembly comprising: a prosthetic valve comprising a frame and at least one actuator coupled to the frame, the actuator operable to move the frame between a radially expanded configuration and a radially compressed configuration; and at least one actuator assembly configured to induce an electromagnetic field, wherein the electromagnetic field releasably couples the at least one actuator assembly to the at least one actuator.

Example 19. The assembly of any example herein, particularly example 18, wherein the at least one actuator assembly comprises a radially outer member and a radially inner member.

Example 20. The assembly of any example herein, particularly example 19, wherein the radially outer member is configured to induce the electromagnetic field.

Example 21. The assembly of any example herein, particularly example 19, wherein the radially inner member is configured to induce the electromagnetic field.

Example 22. The assembly of any example herein, particularly example 21, wherein a distal end of the radially outer member includes a non-circular bore that extends over an outer surface of the at least one actuator when the at least one actuator assembly is coupled to the prosthetic valve.

Example 23. The assembly of any example herein, particularly example 22, wherein the radially outer member is configured to rotate the at least one actuator upon rotation of the radially outer member.

Example 24. The assembly of any example herein, particularly example 23, wherein the radially inner member has a cylindrical end portion that extends into a cylindrical bore of the at least one actuator when the at least one actuator assembly is coupled to the prosthetic valve.

Example 25. The assembly of any example herein, particularly any one of examples 18-24, further comprising a handle coupled to the at least one actuator assembly.

Example 26. The assembly of any example herein, particularly example 25, wherein the handle comprises an input device electrically operatively coupled to the at least one actuator assembly, wherein the input device is configured to magnetize and demagnetize the at least one actuator assembly.

Example 27. The assembly of any example herein, particularly example 26, further comprising one or more wires electrically operatively coupled to the at least one actuator assembly and the input device, wherein the one or more wires extend through a lumen of the at least one actuator assembly.

Example 28. The assembly of any example herein, particularly either example 26 or example 27, wherein the handle comprises one or more visual indicators to indicate whether the at least one actuator assembly is magnetized or demagnetized and/or whether the at least one actuator assembly is in an engaged state or a disengaged state with the at least one actuator.

Example 29. The assembly of any example herein, particularly any one of examples 25-28, wherein the assembly comprises a plurality of actuator assemblies.

Example 30. The assembly of any example herein, particularly example 29, wherein each actuator assembly further comprises a sensor configured to detect a resistive force exerted by the actuator on the actuator assembly to resist rotation.

Example 31. The assembly of any example herein, particularly example 30, wherein the handle further comprises an indicator configured to indicate a curvature of an implantation site of the prosthetic valve based on the resistive forces detected by the sensors.

Example 32. An assembly comprising: a prosthetic valve having a radially expandable and compressible frame, a valvular structure disposed within the frame and configured to regulate a flow of blood through the frame in one direction, and at least one actuator operatively coupled to the frame, wherein the actuator is configured to be rotated, via rotation of a head thereof, to radially expand or compress the frame; and a delivery device comprising a driver member configured to form a releasable electromagnetic connection with the actuator and rotate the actuator upon rotation of the driver member.

Example 33. The assembly of any example herein, particularly example 32, wherein the delivery device further comprises a sleeve member, wherein the driver member is at least partially disposed within the sleeve member.

Example 34. The assembly of any example herein, particularly example 32, wherein the delivery device further comprises an inner support member, wherein the inner support member is at least partially disposed within the driver member.

Example 35. The assembly of any example herein, particularly any one of examples 32-34, wherein the delivery device further comprises a handle coupled to the driver member.

Example 36. The assembly of any example herein, particularly example 35, wherein the handle comprises an input device electrically operatively coupled to the driver member, wherein the input device is configured to magnetize and demagnetize the driver member.

Example 37. The assembly of any example herein, particularly example 36, further comprising one or more wires electrically operatively coupled to the driver member and the input device, wherein the one or more wires extend through a lumen of the driver member.

Example 38. The assembly of any example herein, particularly either example 36 or example 37, wherein the handle comprises one or more visual indicators to indicate whether the driver member is magnetized or demagnetized and/or whether the driver member is in an engaged state or a disengaged state with the actuator.

Example 39. The assembly of any example herein, particularly any one of examples 32-38, wherein the delivery device comprises a plurality of actuator assemblies.

Example 40. The assembly of any example herein, particularly example 39, wherein each actuator assembly further comprises a sensor configured to detect a resistive force exerted by the actuator on the driver member to resist rotation.

Example 41. The assembly of any example herein, particularly example 40, wherein the delivery device further comprises an indicator configured to indicate a curvature of an implantation site of the prosthetic valve based on the resistive forces detected by the sensors.

Example 42. The assembly of any example herein, particularly any one of examples 32-41, wherein the driver member includes an electromagnet configured to induce an electromagnetic field.

Example 43. A delivery apparatus for a prosthetic valve comprising a mechanically actuatable frame comprising a plurality of actuator members, the actuator members configured to be rotated to radially expand or compress the frame, the delivery apparatus comprising: a plurality of actuator assemblies configured to rotate the actuator members of the prosthetic valve, each actuator assembly comprising a rotatable driver member and a sensor electrically coupled to the driver member, wherein the sensor is configured to detect a resistive force exerted by the actuator member on the driver member during rotation.

Example 44. The delivery apparatus of any example herein, particularly example 43, further comprising an indicator configured to indicate a curvature of an implantation site of the prosthetic valve based on the resistive forces detected by the sensors.

Example 45. The delivery apparatus of any example herein, particularly either example 43 or example 44, wherein each actuator assembly is configured induce an electromagnetic field to form a releasable electromagnetic connection with an actuator of the frame and rotate the actuator upon rotation of the driver member.

Example 46. The delivery apparatus of any example herein, particularly example 45, further comprising an inner support member at least partially disposed within the driver member.

Example 47. The delivery apparatus of any example herein, particularly example 46, wherein the inner support member is configured to induce the electromagnetic field.

Example 48. The delivery apparatus of any example herein, particularly example 45, wherein the driver member is configured to induce the electromagnetic field.

Example 49. The delivery apparatus of any example herein, particularly example 48, further comprising an outer sleeve member, wherein the driver member is at least partially disposed within the outer sleeve member.

Example 50. The delivery apparatus of any example herein, particularly any one of examples 43-49, further comprising a handle coupled to the plurality of actuator assemblies.

Example 51. The delivery apparatus of any example herein, particularly example 50, wherein the handle comprises an input device operatively coupled to the plurality of actuator assemblies, wherein the input device is configured to magnetize and demagnetize the plurality of actuator assemblies.

Example 52. The delivery apparatus of any example herein, particularly example 51, further comprising one or more wires electrically operatively coupled to the plurality of actuator assemblies and the input device, wherein the one or more wires extend through a lumen of each of the plurality of actuator assemblies.

Example 53. The delivery apparatus of any example herein, particularly either example 51 or example 52, wherein the handle comprises one or more visual indicators to indicate whether the plurality of actuator assemblies are magnetized or demagnetized and/or whether the plurality of actuator assemblies is in an engaged state or a disengaged state with the actuator members.

Example 54. An assembly comprising: a prosthetic valve comprising a frame and at least one actuator coupled to the frame, the actuator operable to move the frame between a radially expanded configuration and a radially compressed configuration; and a plurality of actuator assemblies, wherein each actuator assembly comprises a driver member configured to rotate the actuator upon rotation of the driver member and a sensor electrically coupled to the driver member, the sensor configured to detect a resistive force exerted by the actuator on the driver member to resist rotation.

Example 55. The assembly of any example herein, particularly example 54, wherein each actuator assembly is configured induce an electromagnetic field to form a releasable electromagnetic connection with the actuator of the frame and rotate the actuator upon rotation of the driver member.

Example 56. The assembly of any example herein, particularly example 55, further comprising an inner support member at least partially disposed within the driver member.

Example 57. The assembly of any example herein, particularly example 56, wherein the inner support member is configured to induce the electromagnetic field.

Example 58. The assembly of any example herein, particularly example 55, wherein the driver member is configured to induce the electromagnetic field.

Example 59. The assembly of any example herein, particularly example 58, further comprising an outer sleeve member, wherein the driver member is at least partially disposed within the outer sleeve member.

Example 60. The assembly of any example herein, particularly any one of examples 54-59, further comprising a handle coupled to the plurality of actuator assemblies.

Example 61. The assembly of any example herein, particularly example 60, wherein the handle comprises an input device operatively coupled to the plurality of actuator assemblies, wherein the input device is configured to magnetize and demagnetize the plurality of actuator assemblies.

Example 62. The assembly of any example herein, particularly example 61, further comprising one or more wires operatively coupled to the plurality of actuator assemblies and the input device, wherein the one or more wires extend through a lumen of each of the plurality of actuator assemblies.

Example 63. The assembly of any example herein, particularly either example 61 or example 62, wherein the handle comprises one or more visual indicators to indicate whether the plurality of actuator assemblies are magnetized or demagnetized and/or whether the plurality of actuator assemblies is in an engaged state or a disengaged state with the at least one actuator.

Example 64. The assembly of any example herein, particularly any one of examples 60-63, wherein the handle comprises an indicator configured to indicate a curvature of an implantation site of the prosthetic valve based on the resistive forces detected by the sensors.

Example 65. A method of implanting a prosthetic valve comprising: magnetically coupling a driver of an actuator assembly of a delivery device to an actuator of the prosthetic valve; delivering the prosthetic valve to an implantation location within a patient's body; radially expanding the prosthetic valve to a functional size; and releasing the driver from the actuator.

Example 66. The method of any example herein, particularly example 65, wherein magnetically coupling the driver to the actuator comprises positioning the driver adjacent to the actuator and inducing an electromagnetic field with the driver.

Example 67. The method of any example herein, particularly example 65, wherein magnetically coupling the driver to the actuator comprises positioning the driver adjacent to the actuator and inducing an electromagnetic field with a support member of the actuator assembly disposed at least partially within the driver.

Example 68. The method of any example herein, particularly either example 66 or example 67, wherein releasing the driver from the actuator comprises turning off the electromagnetic field.

Example 69. The method of any example herein, particularly any one of examples 65-68, wherein radially expanding the prosthetic valve comprises rotating the driver.

Example 70. A delivery apparatus of any example herein, particularly any one of examples 1-69, wherein the delivery apparatus is sterilized.

Example 71. A prosthetic valve of any example herein, particularly any one of examples 1-69, wherein the prosthetic valve is sterilized.

The features described herein with regard to any example can be combined with other features described in any one or more of the other examples, unless otherwise stated. For example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery apparatus for implanting a prosthetic valve comprising a mechanically actuatable frame comprising at least one actuator member, the actuator member configured to be rotated to radially expand or compress the frame, the delivery apparatus comprising:
at least one actuator assembly configured to actuate the actuator member of the prosthetic valve, the actuator assembly comprising an outer sleeve member having a lumen and an inner driver member coaxially disposed within the lumen of the sleeve member, the driver member being rotatable relative to the sleeve member, wherein the actuator assembly is configured to be transitioned between an engaged state and a disengaged state with the prosthetic valve, and wherein the driver member is configured to be selectively magnetically coupled to the actuator member in the engaged state.

2. The delivery apparatus of claim 1, wherein the driver member includes an electromagnet configured to induce an electromagnetic field.

3. The delivery apparatus of claim 2, further comprising a handle coupled to the at least one actuator assembly, wherein the handle comprises an input device operatively coupled to the driver member, wherein the input device is configured to magnetize and demagnetize the electromagnet.

4. The delivery apparatus of claim 3, further comprising one or more wires electrically operatively coupled to the electromagnet and the input device, wherein the one or more wires extend through a lumen of the driver member.

5. The delivery apparatus of claim 3, wherein the handle comprises one or more visual indicators to indicate whether the electromagnet is magnetized or demagnetized and/or whether the at least one actuator assembly is in the engaged state or the disengaged state.

6. An assembly comprising:
a prosthetic valve comprising a frame and at least one actuator coupled to the frame, the actuator operable to move the frame between a radially expanded configuration and a radially compressed configuration; and
at least one actuator assembly configured to induce an electromagnetic field, wherein the electromagnetic field releasably couples the at least one actuator assembly to the at least one actuator.

7. The assembly of claim 6, wherein the at least one actuator assembly comprises a radially outer member and a radially inner member, wherein at least one of the radially outer member and the radially inner member is configured to induce the electromagnetic field.

8. The assembly of claim 7, wherein a distal end of the radially outer member includes a non-circular bore that extends over an outer surface of the at least one actuator when the at least one actuator assembly is coupled to the prosthetic valve.

9. The assembly of claim 7, wherein the radially outer member is configured to rotate the at least one actuator upon rotation of the radially outer member.

10. The assembly of claim 7, wherein the radially inner member has a cylindrical end portion that extends into a cylindrical bore of the at least one actuator when the at least one actuator assembly is coupled to the prosthetic valve.

11. The assembly of claim 6, further comprising a handle coupled to the at least one actuator assembly.

12. The assembly of claim 11, wherein the handle comprises an input device electrically operatively coupled to the at least one actuator assembly, wherein the input device is configured to magnetize and demagnetize the at least one actuator assembly.

13. The assembly of claim 12, further comprising one or more wires electrically operatively coupled to the at least one actuator assembly and the input device, wherein the one or more wires extend through a lumen of the at least one actuator assembly.

14. The assembly of claim 12, wherein the handle comprises one or more visual indicators to indicate whether the at least one actuator assembly is magnetized or demagnetized and/or whether the at least one actuator assembly is in an engaged state or a disengaged state with the at least one actuator.

15. A delivery apparatus for a prosthetic valve comprising a mechanically actuatable frame comprising a plurality of actuator members, the actuator members configured to be rotated to radially expand or compress the frame, the delivery apparatus comprising:
a plurality of actuator assemblies configured to rotate the actuator members of the prosthetic valve, each actuator assembly comprising a rotatable driver member and a sensor electrically coupled to the driver member, wherein the sensor is configured to detect a resistive force exerted by the actuator member on the driver member during rotation.

16. The delivery apparatus of claim 15, further comprising an indicator configured to indicate a curvature of an implantation site of the prosthetic valve based on the resistive forces detected by the sensors.

17. The delivery apparatus of claim 15, further comprising a handle coupled to the plurality of actuator assemblies, wherein the handle comprises an input device operatively coupled to the plurality of actuator assemblies, wherein the input device is configured to magnetize and demagnetize the plurality of actuator assemblies.

18. The delivery apparatus of claim 17, further comprising one or more wires electrically operatively coupled to the plurality of actuator assemblies and the input device, wherein the one or more wires extend through a lumen of each of the plurality of actuator assemblies.

19. The delivery apparatus of claim 17, wherein the handle comprises one or more visual indicators to indicate whether the plurality of actuator assemblies are magnetized or demagnetized and/or whether the plurality of actuator assemblies is in an engaged state or a disengaged state with the actuator members.

20. A method of implanting a prosthetic valve comprising:
magnetically coupling a driver of an actuator assembly of a delivery device to an actuator of the prosthetic valve;
delivering the prosthetic valve to an implantation location within a patient's body;
radially expanding the prosthetic valve to a functional size; and
releasing the driver from the actuator.
